(19) United States
(12) Patent Application Publication
(10) Pub. No.: US 2026/0021240 A1
(43) Pub. Date: Jan. 22, 2026

(54) NEGATIVE PRESSURE WOUND THERAPY DEVICE

(71) Applicant: GENADYNE BIOTECHNOLOGIES, INC., Hicksville, NY (US)

(72) Inventors: Chien Ming GOH, Great Neck, NY (US); Shahzad Saad PIRZADA, Old Westbury, NY (US)

(73) Assignee: GENADYNE BIOTECHNOLOGIES, INC., Hicksville, NY (US)

(21) Appl. No.: 19/272,645

(22) Filed: Jul. 17, 2025

Related U.S. Application Data
(60) Provisional application No. 63/672,916, filed on Jul. 18, 2024.

(51) Int. Cl. A61M 1/00

(52) U.S. Cl. CPC A61M 1/962; A61M 2205/52; A61M 2205/8206

(57) ABSTRACT

At least one embodiment relates to a negative pressure wound therapy device for use with a negative pressure wound therapy pump. The device comprises a housing, a microprocessor, a memory, a transceiver, and at least one fluid channel coupled to the housing. The least one fluid channel is configured to deliver fluid to a wound site and wherein the at least one microcontroller is configured to control a negative pressure wound therapy pump. In at least one embodiment there is a positive pressure pump, wherein the positive pressure pump is configured to deliver fluid in the fluid channel to the wound site. The positive pressure pump is disposed in the housing.

### CROSS REFERENCE TO RELATED APPLICATIONS

This application is a non-provisional application that hereby claims priority from provisional application Ser. No. 63/672,916 filed on Jul. 18, 2024, the disclosure of which is hereby incorporated herein by reference in its entirety.

### BACKGROUND OF THE INVENTION

At least one embodiment relates to a negative pressure wound therapy device add-on which is configured to provide positive pressure fluid into a wound site. The device is configured to work with a negative pressure wound therapy device which is configured to remove a fluid from a wound site. The additional device is configured to provide positive pressure and fluid to that site while controlling a negative pressure wound therapy device as well to coordinate the fluid being input into the wound site.

### SUMMARY

At least one embodiment relates to a negative pressure wound therapy device for use with a negative pressure wound therapy pump. The device comprises a housing, a microprocessor, a memory, a transceiver, and at least one fluid channel coupled to the housing. The least one fluid channel is configured to deliver fluid to a wound site and wherein the at least one microcontroller is configured to control a negative pressure wound therapy pump.

In at least one embodiment there is a positive pressure pump, wherein the positive pressure pump is configured to deliver fluid in the fluid channel to the wound site. The positive pressure pump is disposed in the housing. There can be at least one communication cable, wherein the communication cable is coupled to the housing at a first end and to a negative pressure pump at a second end.

There can be at least one additional communication cable, wherein the additional communication cable is configured to control at least one additional positive pressure pump. The device can include a battery and/or a separate power supply. In at least one embodiment, the microprocessor is configured to send signals along said at least one communication cable, wherein said microprocessor is configured to control the negative pressure wound therapy pump. The device can include at least one screen such as a touch screen. In at least one embodiment, the screen has a menu for controlling said microprocessor, wherein the menu has a list of commands to control the negative pressure wound therapy pump. In at least one embodiment, the device further comprises a remote control. In at least one embodiment there is a method for controlling a negative pressure pump. This process can include the steps of connecting a controller to a negative pressure wound therapy pump. This process can include using a microprocessor to send commands to said negative pressure wound therapy pump to control a pressure level of the negative pressure pump. Next there can be a step of recording a pressure level of the negative pressure wound therapy pump. Next, there can be the step of reading a flow rate of fluid through the negative pressure wound therapy pump. Next, there can be the step of controlling a positive pressure pump to pump fluid to a wound site. Another step can comprise determining a flow rate of the positive pressure pump. Next, there can be the step of determining a pressure at a wound site. The next step includes coupling a positive pressure pump to a controller. housing.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1A is a perspective view of a negative pressure device;

FIG. 1B is a front view of the negative pressure wound therapy device;

FIG. 1C is a side view of the negative pressure wound therapy device;

FIG. 1D is a perspective view of the negative pressure wound therapy device;

FIG. 2 is a layout of a first embodiment;

FIG. 3 is a layout of another embodiment;

FIG. 4A is a view of another embodiment;

FIG. 4B is a view of another embodiment;

FIG. 5A is a schematic block diagram of the electronic components of the negative pressure wound therapy controller device;

FIG. 5B is a schematic block diagram of the electronic components of the computer devices used in the network controlling the negative pressure wound therapy device;

FIG. 5C is a schematic block diagram of the computer network for controlling the negative pressure wound therapy device controller; and

FIG. 6 is a flow chart of the process for controlling the negative pressure wound therapy device.

### DETAILED DESCRIPTION

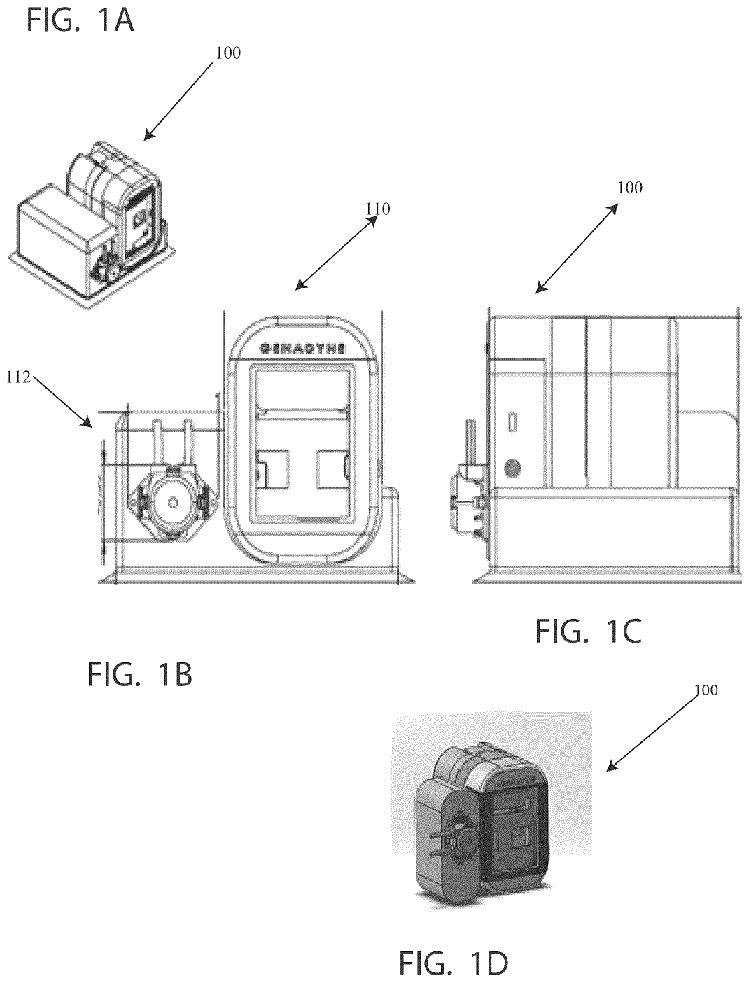

FIG. 1A is a perspective view of a negative pressure controller device 100 which includes a controller 110 as well as a pump 112. FIG. 1B is a front view of the negative pressure controller device 100 which shows the controller 110 and the pump 112. FIG. 1C is a side view of the negative pressure controller device 100, while FIG. 1D is a perspective view of the negative pressure controller device 100 including the controller 110 and the pump 112 as well.

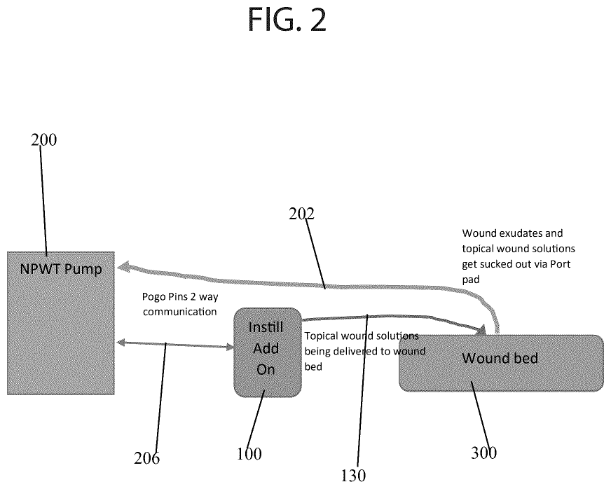

FIG. 2 is a layout of a first embodiment which shows a negative pressure wound therapy pump 200 a wound pad 300 as well as the negative pressure controller device 100. This controller 110 with device 100 is configured to be in communication via either wireless communication or via a wireline 120 in communication with the negative pressure wound therapy pump 200. This wound therapy pump can be in the form of a peristatic pump. The device 100 includes at least one feed line 140 which is a fluid line connecting the pump 112 with the wound bed 300. The feed line 140 is configured to provide fluid in a positive pressure feed to feed either saline or a healing fluid into the wound bed 300. The wound bed 300 can include bandages as well as a sealing cover which is configured to seal over a wound and allow for the positive pressure of a fluid being pumped into the wound bed 300.

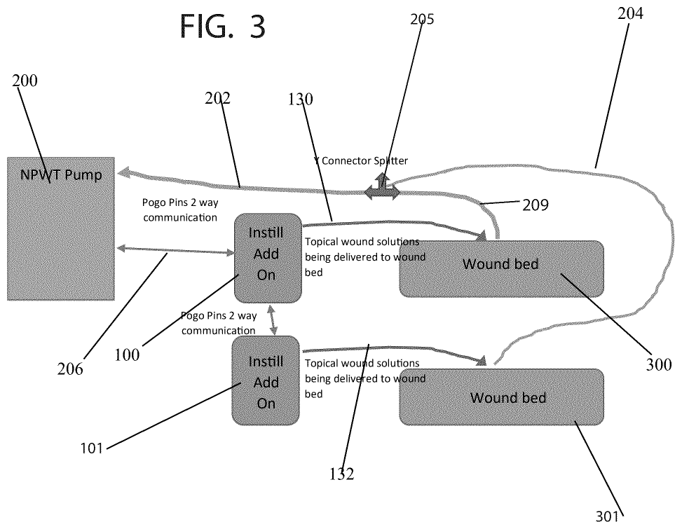

FIG. 3 is a layout of another embodiment which includes a negative pressure wound therapy controller device 100 as well as an additional negative pressure wound therapy controller device 101. While a single negative pressure wound therapy device 200 is provided, two different wound beds 300 and 301 are provided as well as two separate controller devices 100 and 101 are provided so that a single negative pressure wound therapy device 200 can be used for providing negative pressure to two different wound sites 300 and 301 through two different negative pressure lines 204 and 209 which meet at the connector 205 and which then leads to fluid being fed into line 202.

There are fluid lines 130 and 132 which are configured to allow fluid to flow from the different controllers/pump devices 100 and 101 to the respective wound beds 300 and 301. Either one of the add on devices 100 and 101 can control the negative pressure pump 200 via a communication line 206. For example, in at least one embodiment device 100 controls both the negative pressure pump 200 as well as the additional add on device 101 in a master/slave relationship. Therefore, the user who wishes to control the feed of fluid into the wound site can control a single device 100 which then controls the additional devices. Alternatively, each of the two different add on devices 100 and 101 can be separately controlled so that each wound site can 300 and 301 can have separate levels of positive pressure put into the wound sites.

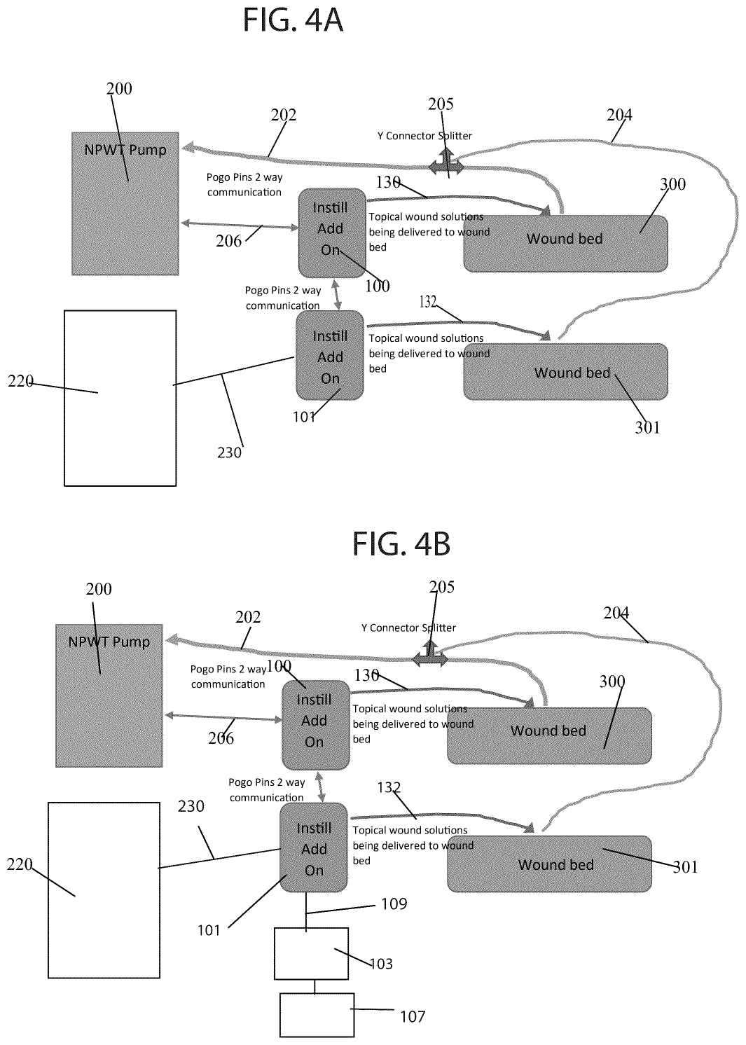

FIG. 4A is a view of another embodiment. This embodiment includes all of the same elements of the previous embodiment in FIG. 3 but also includes an additional negative pressure pump 220 which is configured to work separately from negative pressure pump 200. This negative pressure pump 220 is in communication with/coupled to controller of device 101 via communication line 230. Therefore, in this embodiment, pump 220 is controlled by device 101 while pump 200 is controlled by device 100. However, a single controller such as a controller 110 with device 100 can also control the other controller of device 101 so that a single controller of device 100 can control all of the devices such as pump/pump device 200, pump/pump device 220, and the controller 110 of device 101.

FIG. 4B is a view of another embodiment which shows all of the components shown in FIG. 4A, but it also includes an additional reservoir 103. Reservoir 103 is configured to allow for fluid to flow into devices 100 and 101 via line 109, so that healing fluid such as saline, or antibiotic fluid can flow into a wound site from the reservoir 103 through device 101 and 100. The devices 100 and 101 can be connected either in series with reservoir 103 or in parallel with reservoir 103. There is also a heater 107 which is configured to heat the fluid in reservoir 103 so that the solution would be heated before delivery to the wound beds 300 and 301.

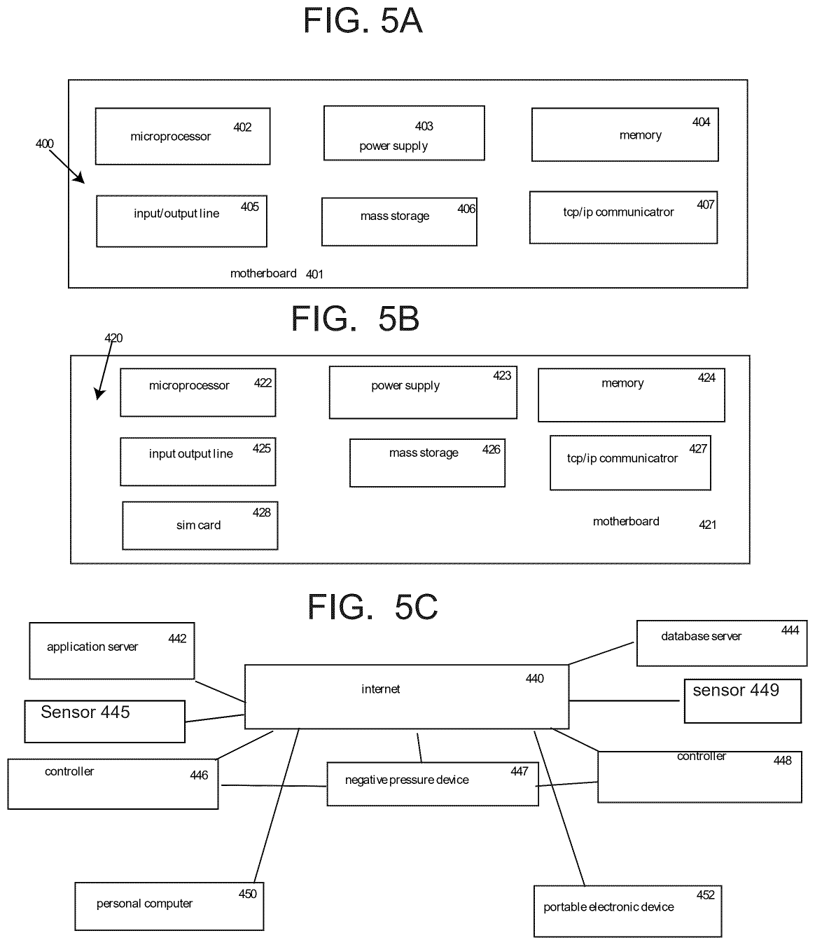

FIG. 5A is a schematic block diagram of the electronic components of the negative pressure wound therapy controller device 100 and 101. With this design which includes a controller 400 which is essentially the same as controller 110 includes a motherboard 401, which is disposed inside of a housing. In addition, coupled to motherboard 401 is a microprocessor 402, a power supply 403 which is configured to provide power to the motherboard. There is also a memory 404, an input output line 405 (communication line), a mass storage device 406 such as a hard drive, and a tcp/ip wireless communicator 407. With this design, the controller device 400/110 can be configured to be programmed via a series of instructions stored in mass storage 406, and then uploaded to memory 404 and then loaded into microprocessor 402 to carry out the series of instructions to control the fluid flow and positive pressure from pump 112 or to control the fluid flow from pump 200 or 201. Communication to all of these devices can be either via a wireless form of communication (via communicator 407) or via line 405.

FIG. 5B is a schematic block diagram of the electronic components of the computer devices used in the network controlling the negative pressure wound therapy device. These components 420 can be integrated into either a server and/or a personal computer and/or a smartphone or other portable device. For example, there is shown a motherboard 421 which is disposed inside of a housing. Coupled to motherboard 421 is a microprocessor 422. There is also a power supply 423 which is configured to provide power to the motherboard 421. There is also a memory 424 coupled to motherboard 421. There is also an input/output line 425, which allows for direct wired communication to the other components in a network (See FIG. 5C). There is a mass storage device 426 which is configured as a hard drive for feeding the instructions/programs for controlling the device(s) 100 and 101 into memory 424 which then feeds into microprocessor 422. There is also a wireless tcp/ip communicator 427 for wireless communication and also a sim card 428 for cellular communication as well.

FIG. 5C is a schematic block diagram of the computer network for controlling the negative pressure wound therapy device controller. With this design there is disclosed an internet 440 which is in communication with an application server 442, and/or a database server 444. The application server and database server can be incorporated into a single server in one embodiment or distributed across a cloud computing network.

There are different controllers/devices 446 and 448 which represent the controllers of devices 100 and 101 respectively. There is also a negative pressure pump/device 447 which represents negative pressure devices 200 and 201 described above. There is also a computer 450 coupled to the network as well as a portable electronic device 452 which is in communication with the network. Sensor(s) 445 and 449 are also positioned around the network at the wound site(s) wound bed(s) 300 and 301, wherein these sensor(s) can be used to detect one of flow rate, pressure at the wound site, pulse rate, temperature at the wound site etc.

Essentially, the controllers/devices 100 and 101 can be remotely controlled either via a web page via computer 450 and/or portable device 452 or via a computer app such as a phone application for controlling these devices. Instructions from computer 450 and/or device 452 can be sent through the internet 440 to application server 442, to instruct these devices 100, 101, 200, 201. In addition, data from these devices including readings from these devices 100, 101, 200, 201 and sensors 445 and 449 can be stored in database server 444. Automatic pre-sets can be stored in database server 444, so that automatic controls can be programmed into the server(s) and then the server(s) can be used for automatic correction of the device(s) 100, 101, 200 and 201 with respect to pressure, flow rate, pulse frequency or any other desired setting.

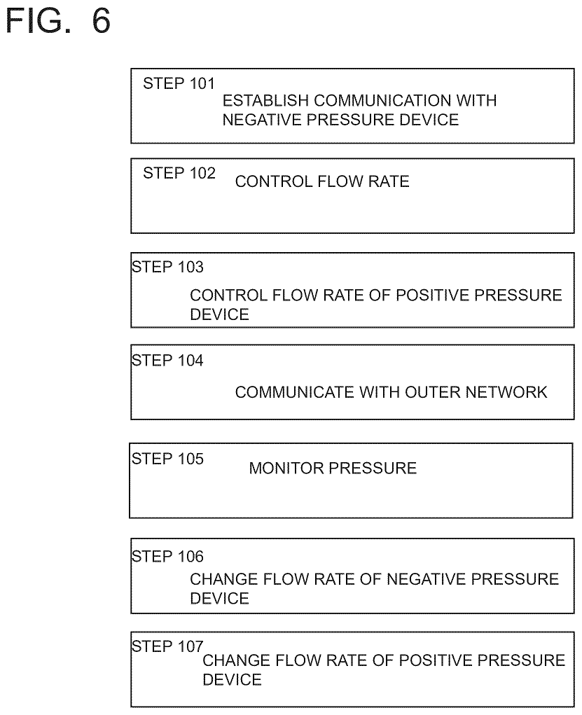

FIG. 6 is a flow chart of the process for controlling the negative pressure wound therapy device. As indicated above, this process is controlled by the controllers which is a combination of the microprocessors (402, 422) in the server(s) (442, 444) as well as the computing devices (450, 452) as well as the controllers (446, 448). For example, the process starts in step 101 which includes establishing a communication between the negative pressure device 200 and the controllers of devices 100 and 101 (see controller 110, 446, 448) of the devices 100, 101. In addition, the communication also is established through the internet with the remote computers 450, 452 and the servers 442, 444. Next, in step 102 the system can control the flow rate of the pump 200. Next, in step 103 the system can control the flow rate of the pump(s) 112 of the devices 100 and 101. Next, once these pumps are working the system can communicate this operation to the outer network such as to the server(s) 442, and 444, as well as to the computing devices 450 and 452. Next, the system can monitor the pressure and other settings, such as flow rate, frequency of pulses, temperature etc., at the wound site 300 and 301 via sensors 445 and 449 at these wound sites. These measurements by the sensors can measure whether there is a rapid drop of pressure at the site, whether there are bubbles created at the site, whether there may be other abnormalities which are recognized as a potential problem for the site.

Next, in step 106 the system can change the settings such as the flow rate of the pump 200 depending on the readings of the sensors at this site. Next, in step 107, the system can change the flow rate and frequency of pulses of the pump of the positive pressure devices 100 and 101 to change the state of the readings at the wound beds 300 and 301.

Thus, there is created an intelligent controller device which is configured to provide positive pressure of fluid flowing into a wound bed 300 and 301 as well as control the negative pressure pump to create a dynamic intelligent pumping system.

Accordingly, while at least one embodiment of the present invention have been shown and described, it is to be understood that many changes and modifications may be made thereunto without departing from the spirit and scope of the invention as defined in the appended claims.

What is claimed is:

1. A negative pressure wound therapy device for use with a negative pressure wound therapy pump, the device comprising:
a housing;
a microprocessor;
a memory;
a transceiver; and
at least one fluid channel coupled to the housing, wherein said at least one fluid channel is configured to deliver fluid to a wound site and wherein said at least one microcontroller is configured to control a negative pressure wound therapy pump.

2. The device as in claim 1, further comprising at least one positive pressure pump, wherein said at least one positive pressure pump is configured to deliver fluid in said fluid channel to the wound site.

3. The device as in claim 2, wherein said positive pressure pump is disposed in said housing.

4. The device as in claim 1, further comprising at least one communication cable, wherein said at least one communication cable is coupled to said housing at a first end and to a negative pressure pump at a second end.

5. The device as in claim 4, further comprising at least one additional communication cable, wherein said at least one additional communication cable is configured to control at least one additional positive pressure pump.

6. The device as in claim 1, further comprising a battery.

7. The device as in claim 5, wherein said microprocessor is configured to send signals along said at least one communication cable, wherein said microprocessor is configured to control the negative pressure wound therapy pump.

8. The device as in claim 1, further comprising at least one screen.

9. The device as in claim 8, wherein said at least one screen is a touch screen.

10. The device as in claim 9, wherein said screen has a menu for controlling said microprocessor, wherein said menu has a list of commands to control said negative pressure wound therapy pump.

11. The device as in claim 1, wherein the device further comprises a remote control.

12. A process for controlling a negative pressure wound therapy pump comprising the steps of:
a) connecting a controller to a negative pressure wound therapy pump;
b) using a microprocessor to send commands to said negative pressure wound therapy pump to control a pressure level of the negative pressure pump;
c) recording a pressure level of the negative pressure wound therapy pump; and
d) reading a flow rate of fluid through the negative pressure wound therapy pump.

13. The process as in claim 12, further comprising the step of controlling a positive pressure pump to pump fluid to a wound site.

14. The process as in claim 13, further comprising the step of determining a flow rate of the positive pressure pump.

15. The process as in claim 14, further comprising the step of determining a pressure at a wound site.

16. The process as in claim 13, further comprising the step of coupling a positive pressure pump to a controller housing.
